(19) United States
(12) Patent Application Publication
(10) Pub. No.: US 2003/0121074 A1
(43) Pub. Date: Jun. 26, 2003

(54) Plant XRCC3 genes and methods of use

(75) Inventors: Gregory D. May, Ardmore, OK (US); Peter R. Beetham, Carlsbad, CA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Boyce Thompson Institute for Plant Research

(21) Appl. No.: 10/162,044

(22) Filed: Jun. 3, 2002

Related U.S. Application Data
(60) Provisional application No. 60/342,817, filed on Dec. 20, 2001.

(51) Int. Cl. A01H 1/00; C12N 9/22; C07H 21/04; C12N 15/82; C12N 5/04

(52) U.S. Cl. 800/286; 435/468; 435/419; 435/199; 435/320.1; 536/23.2

(57) ABSTRACT

The invention relates to isolated nucleic acid molecules encoding plant XRCC3 proteins. Such XRCC3 proteins are believed to be involved in homologous recombination and DNA-repair processes in organisms, particularly plants. The invention provides isolated nucleic acid molecules comprising XRCC3 nucleotide sequences which encode XRCC3 proteins and XRCC3 nucleotide sequences which encode dominant-negative XRCC3 variants. Such XRCC3 nucleotide sequences find use in altering DNA repair, mutation rates and recombination frequencies in both eukaryotic and prokaryotic organisms. Additionally provided are isolated proteins, transformed non-human host cells, and transformed plants, tissues, cells and seeds thereof.

### CROSS-REFERENCE TO RELATED APPLICATIONS

This application claims the benefit of U.S. Provisional Application No. 60/342,817, filed Dec. 20, 2001, which is hereby incorporated herein in its entirety by reference.

### FIELD OF THE INVENTION

The invention relates to the genetic manipulation of plants, particularly to modulating recombination and DNA repair mechanisms in plants.

### BACKGROUND OF THE INVENTION

Homologous recombination plays an important role in the repair of DNA double-strand breaks (DSBs) caused by ionizing radiation or from the breakdown of stalled replication forks. Accurate DSB repair, using the sister chromatid as a template, is necessary for the maintenance of genome stability, and defects in this process can lead to the introduction of mutations, chromosomal translocations, apoptosis, and cancer (Masson et al. (2001) Proc. Natl. Acad. Sci. USA 98:8440-8446).

The RAD51 protein promotes recombination by catalyzing the invasion of the broken ends of the DSB into the intact sister chromatid. RAD51 is a structural and functional homolog of Escherichia coli RecA and forms helical nucleoprotein filaments in which the DNA lies extended and underwound. Filaments form preferentially on tailed duplex DNA substrates that mimic the resected DSBs thought to be present at chromosomal break sites (Mazin et al. (2000) EMBO J. 19:1148-1156; McIlwraith et al. (2000) J. Mol. Biol. 304:151-164). Strand invasion by RAD51 is stimulated by RAD52, RAD54, and RP-A, resulting in the formation of a heteroduplex joint (see Masson et al. (2001) Proc. Natl. Acad. Sci. USA 98:8440-8446 and references 2-11 cited therein). Yeast that are defective in RAD51 exhibit reduced levels of recombination and are sensitive to ionizing radiation, but the cells remain viable. In contrast, disruption of RAD51 in the mouse is lethal (Lim, & Hasty (1996) Mol. Cell. Biol. 16:7133-7143; Tsuzuki et al. (1996) Proc. Natl. Acad. Sci. USA 93:6236-6240). Moreover, inactivation of a RAD51 transgene in chicken cells leads to chromosome fragmentation followed by cell death (Sonoda et al. (1998) EMBO J. 17:598-608). These observations emphasize the essential role that RAD51 and recombinational repair play in normal cellular proliferation. Such an extreme phenotype, however, has not been observed after disruption of RAD52 (Rijkers et al. (1998) Mol. Cell. Biol. 18:6423-6429; Yamaguchi-Iwai et al. (1998) Mol. Cell. Biol. 18:6430-6435) or RAD54 (Bezzubova et al. (1997) Cell 89:185-193; Essers et al. (1997) Cell 89:195-204).

In vertebrates, the RAD51 protein is required for genetic recombination, DNA repair, and cellular proliferation (Masson et al. (2001) Proc. Natl. Acad. Sci. USA 98:8440-8446). Five paralogs of RAD51—RAD51B, RAD51C, RAD51D, XRCC2, and XRCC3—which share at least 25% amino acid sequence identity with RAD51, have been identified and also have been shown to be required for recombination and genome stability (Miller et al. (2001) J. Biol. Chem., Manuscript M108306200, Dec. 13, 2001; Masson et al. (2001) Proc. Natl. Acad. Sci. USA 98:8440-8446).

The RAD51 family members are required for normal levels of recombination and DSB repair (Masson et al. (2001) Proc. Natl. Acad. Sci. USA 98:8440-8446). Whereas cells defective in XRCC2 (irs1) and XRCC3 (irs1SF) are moderately sensitive to x-rays or γ-radiation (2-fold), they display an extreme sensitivity (60- to 100-fold) to DNA cross-linking agents such as cisplatin, nitrogen mustard, or mitomycin C (Liu et al. (1998) Mol. Cell 1:783-793; Tebbs et al. (1995) Proc. Natl. Acad. Sci. USA 92:6354-6358). The mutant cell lines also exhibit a high incidence of spontaneous and mutagen-induced chromosomal aberrations (Cui et al. (1999) Mutat. Res. DNA Repair 434:75-88) and show defects in chromosome segregation (Griffin et al. (2000) Nat. Cell Biol. 2:757-761). Moreover, both irs1 and irs1SF show a significant (100- and 25-fold, respectively) decrease in the frequency of DSB repair by homologous recombination (Johnson et al. (1999) Nature) 401:397-399; Pierce et al. (1999) Genes Dev. 13:2633-2638). While the precise role of XRCC3 in DSB remains unclear, Pierce et al. (1999) Genes Dev. 13:2633-2638) have established, using an XRCC3-deficient hamster cell line, that XRCC3-mediated homologous recombination can reverse DNA damage that is otherwise mutagenic or lethal.

XRCC3 is known to interact with other RAD51 family members and to bind preferentially to single-stranded DNA. When human RAD51C and XRCC3 proteins were overexpressed and purified from baculovirus-infected insect cells, the two proteins co-purified as a complex, a property that reflects their endogenous association observed in HeLa cells (Masson et al. (2001) Proc. Natl. Acad. Sci. USA 98:8440-8446). Furthermore, purified RAD51C-XRCC3 complex was shown to bind to single-stranded, but not duplex DNA, to form protein-DNA networks that could be visualized by electron microscopy (Masson et al. (2001) Proc. Natl. Acad. Sci. USA 98:8440-8446). A recent report indicated that the five RAD51 paralogs-RAD51B, RAD51C, RAD51D, XRCC2, and XRCC3—may exist in a single, large complex and/or several smaller complexes but interestingly the complexes were exclusive of RAD51 (Miller et al. (2001) J. Biol. Chem., Manuscript M108306200, Dec. 13, 2001). However, there is evidence from yeast two-hybrid and co-immunoprecipitation analyses to support that hypothesis that XRCC3 interacts with and/or binds to RAD51 (Liu et al. (1998) Mol. Cell 1:783-793; Schild et al. (2000) J. Biol. Chem. 275:16443-16449).

### SUMMARY OF THE INVENTION

Compositions and methods for altering DNA repair processes and homologous recombination plants and other organisms are provided. Such compositions and methods find use in altering recombination frequencies, and in improving the efficiency of transformation and gene modification, in both eukaryotic and prokaryotic organisms. The compositions comprise isolated nucleic acid molecules comprising nucleotide sequences encoding Arabidopsis XRCC3 (AtXRCC3) proteins, and the proteins encoded by such nucleotide sequences. Further provided are expression cassettes comprising a XRCC3 nucleotide sequence of the invention operably linked to a promoter that drives expression in an organism of interest. The methods involve introducing into an organism an XRCC3 nucleotide sequence of the invention operably linked to a promoter that drives expression in the organism or alternatively introducing XRCC3 proteins into the organism. If decreased expression is desired, the methods can additionally involve co-suppression or antisense suppression.

Transformed non-human host cells, transformed plants, tissues, cells and seeds thereof are provided.

### BRIEF DESCRIPTION OF THE DRAWINGS

FIG. 1 provides the result of ATPase assays with purified AtXRCC3 and demonstrates that AtXRCC3 has ATPase activity that is Mg²⁺ dependent.

FIG. 2 provides the results of ATPase assays and GTPase assays with purified AtXRCC3. FIG. 2A is a graphical depiction of a time course of the ATPase and GTPase activities of AtXRCC3. FIG. 2B illustrates the results of the time course of ATPase and GTPase activity assays with AtXRCC3 as is determined by the accumulation of ³²PO₄.

### DETAILED DESCRIPTION OF THE INVENTION

The invention is drawn to the processes of genetic recombination and DNA repair in living organisms. In particular, the invention provides isolated nucleic acid molecules comprising nucleotide sequences which encode XRCC3 proteins from plants, particularly Arabidopsis, and the isolated proteins encoded by such nucleotide sequences. Such nucleotide sequences find use in plants and other organisms in altering the frequency of recombination and genetic transformation, and the efficiency of gene modification processes such as, for example, chimeraplasty.

Compositions of the invention include nucleotide sequences from genes that encode proteins known as XRCC3 proteins. The amino acid sequence of XRCC3 shares significant homology with the amino acid sequence of RAD51, and accordingly XRCC3 is known as a RAD51 paralog. RAD51 is known to be involved in homologous recombination. In particular, RAD51 promotes homologous recombination by catalyzing the invasion of the broken ends of the double-strand break (DSB) into the intact sister chromatid (Masson et al. (2001) Proc. Natl. Acad. Sci. USA 98:8440-8446).

In particular, the present invention provides for isolated nucleic acid molecules comprising nucleotide sequences encoding the amino acid sequence shown in SEQ ID NO: 2, or the nucleotide sequences encoding the DNA sequences deposited in a bacterial host as Patent Deposit No. PTA-1891. Further provided are polypeptides having an amino acid sequence encoded by a nucleic acid molecule described herein, for example the nucleic acid molecule set forth in SEQ ID NO: 1, the nucleic acid molecule deposited in a bacterial host as Patent Deposit No. PTA-1891, and fragments and variants thereof.

Plasmids containing the nucleotide sequences of the invention were deposited with the Patent Depository of the American Type Culture Collection (ATCC), 10801 University Blvd., Manassas, Va., on May 18, 2000 and assigned Patent Deposit No. PTA-1891. The deposit will be maintained under the terms of the Budapest Treaty on the International Recognition of the Deposit of Microorganisms for the Purposes of Patent Procedure. The deposit was made merely as a convenience for those of skill in the art and are not an admission that a deposit is required under 35 U.S.C. § 112.

The invention encompasses isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably culture medium represents less than about 30%, 20%, 10%, or 5% (by dry weight) of chemical precursors or non-protein-of-interest chemicals.

Fragments and variants of the disclosed nucleotide sequences and proteins encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native XRCC3 protein and hence homologous recombination and/or DNA-repair activity. The biological activity of the native XRCC3 protein is also referred to herein as XRCC3 activity. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins of the invention.

A fragment of an XRCC3 nucleotide sequence that encodes a biologically active portion of an XRCC3 protein of the invention will encode at least 15, 25, 30, 50, 100, 150, 200, 250, or 300 contiguous amino acids, or up to the total number of amino acids present in a fill-length XRCC3 protein of the invention (for example, 304 amino acids for SEQ ID NO: 2, respectively). Fragments of an XRCC3 nucleotide sequence that are useful as hybridization probes or PCR primers generally need not encode a biologically active portion of an XRCC3 protein.

Thus, a fragment of an XRCC3 nucleotide sequence may encode a biologically active portion of an XRCC3 protein, or it may be a fragment that can be used as a hybridization probe or PCR primer using methods disclosed below. A biologically active portion of an XRCC3 protein can be prepared by isolating a portion of one of the XRCC3 nucleotide sequences of the invention, expressing the encoded portion of the XRCC3 protein (e.g., by recombinant expression in vitro), and assessing the activity of the encoded portion of the XRCC3 protein. Nucleic acid molecules that are fragments of an XRCC3 nucleotide sequence comprise at least 16, 20, 50, 75, 100, 150, 200, 250, 300, 350, 400, 450, 500, 550, 600, 650, 700, 750, 800, 850, or 900 nucleotides, or up to the number of nucleotides present in a full-length XRCC3 nucleotide sequence disclosed herein (for example, 915 nucleotides for each of SEQ ID NO: 1).

By “variants” is intended substantially similar sequences. For nucleotide sequences, conservative variants include those sequences that, because of the degeneracy of the genetic code, encode the amino acid sequence of one of the XRCC3 polypeptides of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode an XRCC3 protein of the invention. Generally, variants of a particular nucleotide sequence of the invention will have at least about 70%, generally at least about 75%, 80%, 85%, preferably at least about 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, that is, homologous recombination and/or DNA-repair activity as described herein. Such variants may result from, for example, genetic polymorphism or from human manipulation. Biologically active variants of a native XRCC3 protein of the invention will have at least about 70%, generally at least about 75%, 80%, 85%, preferably at least about 90%, 91%, 92%, 93%, 94%, 95%, 96%, 97%, and more preferably at least about 98%, 99% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants of the XRCC3 proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

Thus, the genes and nucleotide sequences of the invention include both the naturally occurring sequences as well as mutant forms. Likewise, the proteins of the invention encompass both naturally occurring proteins as well as variations and modified forms thereof. Such variants will continue to possess the desired XRCC3 activity; the mutations that will be made in the DNA encoding the variant must not place the sequence out of reading frame and preferably will not create complementary regions that could produce secondary mRNA structure. See, EP Patent Application Publication No. 75,444.

Additionally, the proteins of the invention also encompass fragments and variants that can be in dominant-negative strategies for reducing the biological activity of an XRCC3 and/or its complexes with other proteins, particularly RAD51 family members as described supra. Such dominant-negative XRCC3 proteins of the invention, when expressed in a cell, are capable of reducing or eliminating the biological activity of an XRCC3 and/or its protein complexes therein. That is such dominant-negative XRCC3 proteins confer on the cell, or an organism comprising one or more of said cells, a dominant-negative phenotype. It is recognized that such dominant-negative XRCC3 proteins can be variants of full-length XRCC3 proteins or can be truncated forms or fragments. The invention also encompasses the nucleotide sequences that encode these dominant-negative fragments and variants.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays to assess XRCC3 activity. That is, the XRCC3 activity can be evaluated by one or more methods known in the art including, but not limited to, determining recombination frequency, determining mutation rates, gel shift assays to demonstrate binding to DNA and/or other RAD51 family members, and ATPase activity assays as described herein below.

Variant nucleotide sequences and proteins also encompass sequences and proteins derived from a mutagenic and/or recombinogenic procedure such as DNA shuffling. With such a procedure, one or more different XRCC3 coding sequences can be manipulated to create a new XRCC3 possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the XRCC3 gene of the invention and other known XRCC3 genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The nucleotide sequences of the invention can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire XRCC3 sequences set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Inis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the XRCC3 sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire XRCC3 sequence disclosed herein, or one or more portions thereof, may be used as a probe capable of specifically hybridizing to corresponding XRCC3 sequences and messenger RNAs. To achieve specific hybridization under a variety of conditions, such probes include sequences that are unique among XRCC3 sequences and are preferably at least about 10 nucleotides in length, and most preferably at least about 20 nucleotides in length. Such probes may be used to amplify corresponding XRCC3 sequences from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1X to 2X SSC (20X SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5X to 1X SSC at 55 to 60° C. Exemplary high stringency conditions include hybridization in 50% formamide, 1 M NaCl, 1% SDS at 37° C., and a wash in 0.1X SSC at 60 to 65° C. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6(log M)+0.41(% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-lnterscience, New York). See Sambrook et al. (1989) Molecular Cloning: A Laboratory Manual (2d ed., Cold Spring Harbor Laboratory Press, Plainview, N.Y.).

Thus, isolated sequences that encode of an XRCC3 protein and which hybridize under stringent conditions to the XRCC3 sequences disclosed herein, or to fragments thereof, are encompassed by the present invention.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide or polypeptide sequence, wherein the polynucleotide or polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides or amino acids in length, and optionally can be 30, 40, 50, 100, or longer. Those of skill in the art understand that to avoid a high similarity to a reference sequence due to inclusion of gaps in the polynucleotide or polypeptide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Preferred, non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 10 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

Unless otherwise stated, sequence identity/similarity values provided herein refer to the value obtained using GAP version 10 using the following parameters: % identity using GAP Weight of 50 and Length Weight of 3; % similarity using Gap Weight of 12 and Length Weight of 4, or any equivalent program. By “equivalent program” is intended any sequence comparison program that, for any two sequences in question, generates a global alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 for polypeptides and NWSEAPDNA for polynucleotides (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The XRCC3 amino acid sequence set forth in SEQ ID NO: 2 was analyzed using the MEME system (Version 3.0). See, Bailey and Elkan (1994) Proceedings of the Second International Conference on Intelligent Systems for Molecular Biology (AAAI Press, Menlo Park, Calif.) pp. 28-36; and Bailey and Gribskov (1998) Bioinformatics 14:48-54. The MEME system is a set of tools for discovering patterns in groups of related sequences and for using those patterns to search other sequences for occurrences of the patterns. The MEME system can be used for visualizing the important areas of conservation in a group sequences. The MEME system consists of two tools: MEME (Multiple Expectation Maximization for Motif Elicitation) and MAST (Motif Annotation and Search Tool). MEME is a tool for discovering motifs in groups of sequences. MAST uses the motifs discovered by MEME in a group of related sequences to annotate other sequences. Each sequence in a database of sequences is searched for matches with each of the motifs.

The results of the MEME/MAST analysis revealed that five protein domains or blocks of amino acid sequence are shared between the human, mouse, and AtXRCC3 protein (SEQ ID NO: 2), indicating that these three proteins are related both structurally and functionally. Two of these five domains contain sequence of known function based on studies of other proteins. Motif 1 from the MEME/MAST analysis contains an ATP-binding domain, and Motif 2 contains a zinc-finger domain.

The XRCC3 nucleotide sequences find use in methods for altering homologous recombination and/or DNA repair processes in plants and other organisms. XRCC3 is known to be involved in homologous recombination that can reverse mutagenic or lethal DNA damage in an organism (Pierce et al. (1999) Genes Dev. 13:2633-2638). While the present invention does not depend on a particular mechanism, it is believed that XRCC3 facilitates homologous recombination and/or repair of DSBs in an organism through direct or indirect interactions with several other proteins, including but not limited to RAD51, RAD51B, RAD51C, RAD51D, and XRCC2. To alter homologous recombination and DNA repair in an organism, the organism can be transformed with an XRCC3 nucleotide sequence of the invention, or a fragment or variant thereof.

An alteration in DNA repair in an organism can comprise at least one of change in the DNA of an organism, or at least one cell thereof. Such changes include, but are not limited to, substitutions, additions, deletions, inversions, and other rearrangements. Typically, such an alteration in DNA repair can be determined by monitoring mutation frequency. Methods for monitoring mutation frequency are known in the art and typically involve determining whether a change has occurred in the DNA sequence of one or more genes by monitoring loss, or gain, of a particular function associated with a particular product encoded by the gene. Other methods can be employed, however, to ascertain mutation frequency at the nucleic acid level including, but not limited to, RFLP analysis, PCR, and DNA sequencing. Typically, mutation frequency is assessed by comparing the mutation frequency of an organism that is modified according to the methods of the present invention to a control organism or similar unmodified organism.

The methods of the invention additionally find use in altering recombination frequency in plants and other organisms. By expressing an XRCC3 nucleotide sequence of the invention in a plant or other organism, recombination efficiency can be altered. Decreasing the level or activity of XRCC3 in an organism is expected to increase the integration of exogenous DNA through homologous or homeologous recombination into specific targets within the genome. Thus, the XRCC3 nucleotide sequences can be used to increase integration of foreign DNA into target genes within the genome. Furthermore, the XRCC3 nucleotide sequences can be employed to increase the efficiency of methods of in vivo genetic modification. Such methods are believed to involve recombination, and include, for example, chimeraplasty and gene replacement.

By “exogenous DNA” is intended any nucleic acid molecule that is introduced into a cell. It is recognized that the invention also encompasses nucleic acid molecules comprised of deoxyribonucleotides, ribonucleotides, and combination thereof. Such deoxyribonucleotides and ribonucleotides include, but not limited to, naturally occurring and synthetic form, and derivatives thereof.

By lowering the level or activity of XRCC3 in a plant or other organism, the efficiency of chimeraplasty or gene replacement can be increased. By “efficiency of chimeraplasty” or “efficiency of gene replacement” is intended the proportion of cells or organisms having the desired genetic modification recovered from the total number of cells or organisms used in a chimeraplasty or gene replacement attempt, respectively.

The methods of the invention additionally encompass the use of dominant-negative strategies to reduce a particular biological activity of an XRCC3 within an organism. Such strategies involve the expression in an organism of an XRCC3 nucleotide sequence of the invention, or fragment thereof that encodes a portion of the XRCC3. The methods of the invention additionally encompass nucleotide sequences encoding variants of the XRCC3 proteins of the invention, and fragments thereof, that can be used in dominant-negative strategies to reduce the biological activity of an XRCC3 within an organism or cell thereof. Such dominant-negative strategies are known in the art and can involve the expression of a modified subunit of a multisubunit protein. See, for example, Alani et al. (1997) Mol. Cell Biol. 17:2436-244; Drotschmann et al. (1999) Proc. Natl. Acad. Sci. USA 96:2970-2975; and Wu and Marinus (1994) J. Bact. 176: 5393-5400; all of which are hereby herein incorporated by reference. Generally, such a modified subunit comprises a polypeptide that is able to affect, or interact with, other members of the multisubunit protein complex and thereby reduce, or eliminate, the biological activity of the complex. While the methods of the invention do not depend a particular biological mechanism, typically such a dominant-negative approach will involve the expression of a variant of a XRCC3 protein of the invention that does not possess the complete biological activity of the native protein. It is recognized that such an dominant-negative approach does not depend on eliminating or reducing the expression of native XRCC3 genes in a plant, only that such an approach involves the expression of a variant of an XRCC3 of the invention that is capable of causing a dominant-negative phenotype.

By “dominant-negative phenotype” is intended a phenotype that, when compared to a wild-type phenotype or a previous phenotype of the organism, is substantially altered in a negative manner including, but not limited to, a loss or reduction in a particularly cellular function such as, for example, an enzyme activity (i.e. ATPase activity), homologous recombination, DNA-binding activity and DNA-repair activity repair. Further it is recognized that, while the methods of the invention can be used to negatively affect, through a dominant-negative approach, the cellular activity of an XRCC3 protein, or complex thereof, desired phenotypic changes can result in a organism including, but not limited to, an increase in recombination, an improvement in transformation efficiency and an increase in the efficiency of chimeraplasty.

In an embodiment of the invention, a nucleotide construct comprising an XRCC3 sequence of the invention, or variant or fragment thereof, is introduced into an organism or host cell, particularly a bacterial cell, more particularly an E. coli cell. DNA repair is then monitored in the transformed organism, or host cell, by, for example, determining the mutation rate. Such transformed organisms and host cells find use in producing XRCC3 nucleotide sequences that can be used in dominant-negative strategies to disrupt DNA repair processes in bacteria, plants, and other organisms. Such desired XRCC3 nucleotide sequences encode dominant-negative XRCC3 variants. By “dominant-negative XRCC3 variant” is intended a polypeptide that is capable of conferring a dominant-negative phenotype on a host cell. In particular, the dominant-negative phenotype will impair DNA repair in a host cell or organism. Such an impairment in DNA repair can cause an increase in the mutation rate and/or recombination frequency in a host cell or organism. Thus, the desired XRCC3 nucleotide sequence, which encodes a dominant-negative XRCC3 variant, can be identified by, for example, selecting a host cell with impaired DNA repair as detected by an increase in the mutation rate and/or recombination frequency therein. Such desired XRCC3 nucleotide sequences and the dominant-negative XRCC3 variants encoded thereby find use in methods for altering DNA repair processes, particularly methods for increasing the mutation and/or recombination rates in an organism.

Thus, the invention provides methods for identifying XRCC3 nucleotide sequences which encode dominant-negative XRCC3 variants that are capable of conferring a dominant-negative phenotype on a cell. The invention further provides isolated XRCC3 nucleotide sequences encoding such XRCC3 variants, the dominant-negative XRCC3 variants encoded thereby, and host cells and organisms transformed with such XRCC3 nucleotide sequences. Such transformed host cells and organisms include, but are not limited to, bacteria, yeast, fungi, animals and plants.

The dominant-negative XRCC3 variant of the invention involve the use of an XRCC3 amino acid sequence having at least one amino acid substitution, truncation, internal deletion or insertion. Any XRCC3 nucleotide sequence and any XRCC3 amino acid sequence known the art can be used in the methods of the present invention. Such XRCC3 nucleotide sequences and amino acid sequences include, but are not limited to, GenBank Accession Nos. AB073492, AB062455, XM 050297, XM 053281, and XM 050295. At least one substitution, truncation, internal deletion or insertion can be introduced in the amino acid sequence of an XRCC3 protein by, for example, modifying the nucleotide sequence that encodes the XRCC3 protein using methods known in the art. The modified XRCC3 nucleotide sequence can then be introduced into an organism or host cell according to the methods of the present invention.

For expression in E. coli, the expression cassette can additionally comprise an operably linked promoter. Preferably, such a promoter drives high level gene expression in E. coli, such as, for example, the T5 promoter. The expression cassette can further comprise a nucleotide sequence that encodes an epitope or tag that can be readily detected by immunological or other known methods. Such epitopes or tags, and methods for their use, are known in the art. A nucleotide sequence encoding the epitope or tag can be operably linked to the XRCC3 nucleotide sequence for the transcription of a fusion protein comprising the XRCC3 amino acid sequence and the amino acid sequence of the epitope or tag. Typically, the epitope or tag is N-terminal or C-terminal relative to the XRCC3 amino acid sequence. Such epitopes and tags are known in the art to be useful for the detection and/or purification of fusion proteins.

In another embodiment of the invention, methods are provided for decreasing the level or activity of an XRCC3 protein of the invention in a plant or cell thereof. Plants or cells with decreased XRCC3 protein or activity find use in methods for increasing recombination frequency, increasing mutation rate and increasing the efficiency of chimeraplasty. The level or activity of XRCC3 can be reduced in the plant or cell by, for example, introducing into the plant or cell, a nucleotide construct comprising a promoter that drives expression in a plant operably linked to an XRCC3 nucleotide sequence of the invention. The methods can additionally involve co-suppression, antisense suppression or a dominant-negative strategy to reduce or substantially eliminate the biological activity of XRCC3.

Alternatively, an XRCC3 nucleotide sequence of the invention that encodes an XRCC3 protein that is known to cause a dominant-negative phenotype, can be directly introduced into a plant or other host cell. Such XRCC3 proteins encompass the fragments and variants as discussed supra. Any method for introducing a protein into a plant or other host cell that is known in the art can be employed in the methods of the present invention. For example, a protein can be introduced into a plant by particle bombardment in a manner analogous to that used for the introduction of nucleic acids. See, U.S. Pat. No. 4,945,050. The XRCC3 protein can be associated with or precipitated onto the microprojectiles or microparticles and then bombarded into plant cells. Nucleotide constructs comprising, for example, a chimeraplast or other nucleotide sequence comprising a gene of interest can also be associated with the same microprojectiles or two separate groups of microprojectilesone with the XRCC3 protein and the other with the chimeraplast or nucleotide sequence of interest-an be prepared and then co-bombarded. Alternatively, the plant cells can be bombarded separately with the XRCC3-associated particles and the chimeraplast-associated or nucleotide construct-associated particles.

In another embodiment of the invention, the XRCC3 protein can be produced in an Agrobacterium cell and delivered to a plant cell by the Agrobacterium cell at about the same time as the bacterial cell delivers its Ti plasmid, comprising a gene of interest, to the plant cell. To produce an XRCC3 protein of the invention in Agrobacterium, an Agrobacterium cell can be transformed with an XRCC3 nucleotide sequence of the invention that is operably linked to a promoter that drives expression in the bacterial cell. Methods for transforming Agrobacterium are known in the art and include, but are not limited to, electroporation. Promoters that drive the expression of operably linked nucleotide sequences in Agrobacterium are also known in the art. It is recognized that to facilitate the transfer of the XRCC3 protein into a plant cell, fusion proteins comprising at least a portion of an XRCC3 protein of the invention and at least a portion of one or more additional proteins such as, for example, VirF and VirE2 can be obtained by preparing a nucleotide construct comprising at least a portion of an XRCC3 nucleotide sequence of the invention operable operably linked to a coding sequence for the additional protein or desired portion thereof. The construction of such fusion proteins for transfer by Agrobacterium to a plant cell, and methods of use for such fusion proteins, are known to those of ordinary skill in the art. Generally, such methods involve fusing the protein of interest to the N-terminal end of a VirF, VirE2, or a transport domain thereof. The transport domains of VirF and VirE2 proteins are known to be located in the C-terminal regions of the proteins. See, Vergunst et al. ((2000) Science 290:979-982) and the references cited therein; herein incorporated by reference.

The XRCC3 nucleotide sequences and proteins of the invention find further use in methods for improving transformation efficiency. By “improving transformation efficiency” is intended an increase in the recovery of transformed cells, tissues, organs, or organisms from a transformation attempt. The methods of the invention involve introducing one or more XRCC3 nucleotide sequences or proteins into a host cell. Such host cells can provide improved transformation efficiency in a transformation attempt. Typically, the XRCC3 nucleotide sequences or proteins will be introduced into a host cell prior to, or concomitantly, with a nucleotide sequence of interest to improve transformation efficiency with respect to the nucleotide sequence of interest. In particular, the methods can increase the recovery of stably transformed cells, tissues, organs, or organisms in a transformation attempt. Thus, the invention further provides improved methods for transforming organisms, particularly plants.

While invention does not depend on a particularly biological or genetic mechanism, it is recognized that altering recombination and DNA repair in an organism can affect cellular processes that are involved in the stable incorporation of a nucleotide construct of interest, or at least one nucleotide thereof, into the genome of the cell. Further, it is recognized that reducing, or otherwise inhibiting, DNA repair in an organism can improve the efficiency of genetic transformation methods, such as, for example, chimeraplasty, which are believed to involve circumventing the DNA repair system of a host cell.

In an embodiment of the invention, a plant is stably transformed with a nucleotide construct comprising an XRCC3 nucleotide sequence of the invention operably linked to a promoter that drives expression in a plant cell. Such a plant finds use in methods for improving transformation efficiency. Preferably, the promoter drives expression in plant cells that are targeted for transformation with a nucleotide sequence of interest. More preferably, the promoter is a tissue-preferred or chemical-regulated promoter. Such methods can additionally involve sense or antisense suppression of XRCC3 expression in the plant of interest. Alternatively, the methods can also involve a dominant-negative strategy as described supra to lower the activity of XRCC3 and/or its protein complexes in the plant of interest.

The XRCC3 sequences of the invention are provided in expression cassettes for expression in the plant or other organism of interest. For XRCC3 coding sequences, the cassette will include 5′ and 3′ regulatory sequences operably linked to a XRCC3 sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

The expression cassettes are provided with a plurality of restriction sites for insertion of the XRCC3 coding sequence to be under the transcriptional regulation of the regulatory regions. The expression cassette may additionally contain selectable marker genes.

The expression cassette will include in the 5′-3′ direction of transcription, a transcriptional and translational initiation region, a XRCC3 DNA sequence of the invention, and a transcriptional and translational termination region functional in plants. The transcriptional initiation region, the promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a “chimeric gene” comprises a coding sequence operably linked to a transcription initiation region that is heterologous to the coding sequence, or any combination of a promoter with a coding sequence that is not identical to the structure of a native, unmodified gene.

While it may be preferable to express the sequences using heterologous promoters, the native promoter sequences may be used. Such constructs would change expression levels of XRCC3 in the plant or plant cell. Thus, the phenotype of the plant or plant cell is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed plant. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picomavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced XRCC3 expression within a particular plant tissue. Tissue-preferred promoters include, but are not limited to, those described by Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1 993) Plant Mot Biol. 23(6): 1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 ( Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (I1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1 984) EMBO J. 3:2717-2722), and ballistic particle acceleration (see, for example, Sanford et al., U.S. Pat. No. 4,945,050; Tomes et al., U.S. Pat. No. 5,879,918S; Tomes et al., U.S. Pat. No. 5,8 86,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Annu. Rev. Genet. 22:421-477; Sanford et al. (1987) Particulate Science and Technology 5:27-37 (onion); Christou et al. (1 988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

The methods of the invention involve introducing a nucleotide construct into a plant. By “introducing” is intended presenting to the plant the nucleotide construct in such a manner that the construct gains access to the interior of a cell of the plant. The methods of the invention do not depend on a particular method for introducing a nucleotide construct to a plant, only that the nucleotide construct gains access to the interior of at least one cell of the plant. It is recognized that certain embodiments of the invention do not depend on the stable incorporation of the XRCC3 nucleotide sequences of the invention into the genome of an organism.

Methods for introducing nucleotide constructs into plants are known in the art including, but not limited to, stable transformation methods, transient transformation methods, and virus-mediated methods. By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

The nucleotide constructs of the invention can be introduced into plants by contacting plants with a virus or viral nucleic acids. Generally, such methods involve incorporating a nucleotide construct of the invention within a viral DNA or RNA molecule. It is recognized that an XRCC3 of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

It is recognized that with these nucleotide sequences, antisense constructions, complementary to at least a portion of the messenger RNA (mRNA) for the XRCC3 sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity. See, U.S. Pat. Nos. 5,283,184 and 5,034,323; herein incorporated by reference.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

Furthermore, it is recognized that the methods of the invention may employ a nucleotide construct that is capable of directing, in a transformed plant, the expression of at least one protein, or at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods of the invention can employ a nucleotide construct that is not capable of directing, in a transformed plant, the expression of a protein or an RNA.

In addition, it is recognized that methods of the present invention do not depend on the incorporation of the entire nucleotide construct into the genome, only that the plant or cell thereof is altered as a result of the introduction of the nucleotide construct into a cell. In one embodiment of the invention, the genome may be altered following the introduction of the nucleotide construct into a cell. For example, the nucleotide construct, or any part thereof, can incorporate into the genome of the plant. For the present invention, alterations to the genome include, but are not limited to, additions, deletions, and substitutions of nucleotides in the genome. While the methods of the present invention do not depend on additions, deletions, or substitutions of any particular number of nucleotides, it is recognized that such additions, deletions, or substitutions comprise at least one nucleotide.

The nucleotide constructs of the invention also encompass nucleotide constructs -that may be employed in methods for altering or mutating a genomic nucleotide sequence in an organism, including, but not limited to, chimeraplasts, chimeric vectors, chimeric mutational vectors, chimeric repair vectors, mixed-duplex oligonucleotides, self-complementary chimeric oligonucleotides, and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

The invention further provides host cells transformed with at least one of the XRCC3 nucleotide sequences of the invention. The host cells of the invention can be from any organism including, but not limited to, bacteria, fungi, animals and plants. A nucleotide construct comprising an XRCC3 nucleotide sequence of the invention can be introduced into a host cell by any transformation methods known in the art. Such an introduced nucleotide construct can be stably integrated in the genome of the host cell or be present within the host cell in non-integrated form such as, for example, a plasmid, a cosmid, an artificial chromosome, or other vector. Expression cassettes can be constructed which include the nucleotide constructs of interest operably linked with the transcriptional and translational regulatory signals for expression of the nucleotide construct within the desired host cell.

Transcriptional and translational regulatory signals include, but are not limited to, promoters, transcriptional initiation start sites, operators, activators, enhancers, other regulatory elements, ribosomal binding sites, an initiation codon, termination signals, and the like. See, for example, U.S. Pat. No. 5,039,523; U.S. Pat. No. 4,853,331; EPO 0480762A2; Sambrook et al. supra; Molecular Cloning, a Laboratory Manual, Maniatis et al. (eds) Cold Spring Harbor Laboratory, Cold Spring Harbor, N.Y. (1982); Advanced Bacterial Genetics, Davis et al. (eds.) Cold Spring Harbor Laboratory, Cold Spring Harbor, N.Y. (1980); and the references cited therein.

The non-human host cells of the invention can be used as a source of XRCC3 proteins for the isolation or purification of such proteins. If desired and using methods known to those of ordinary skill in the art, expression systems can be designed in such a manner to cause the XRCC3 proteins to be secreted outside the cytoplasm of a bacterium, such as, for example, the gram-negative bacterium, E. coli. Advantages of having XRCC3 secreted include, but are not limited to, (1) a reduction in, or avoidance of, potential cytotoxic effects of XRCC3, and (2) an improvement in the efficiency of purification of XRCC3. By “improvement in the efficiency of purification” is intended an improvement in at least one aspect of protein purification, including by not limited to, decreased cost of purification of a unit amount of protein, increased recovery of protein per purification attempt, increased recovery of active protein per purification attempt, and increased protein yield per bacterial cell or volume of culture broth. In addition, the invention encompasses fusion proteins comprising an XRCC3 of the invention and a epitope or tag that can be used to facilitate purification and/or detection of such a fusion protein. Such epitopes or tags, and methods of use, are known in the art and include, for example, the polyhistidine-tag (his-tag).

XRCC3 can be modified for secretion in E. coli by, for example, fusing an appropriate E. coli signal peptide to the amino-terminal end of the XRCC3 protein. Signal peptides recognized by E. coli can be found in proteins already known to be secreted in E. coli, such as, for example the OmpA protein (J. Ghrayeb et al. (1984) EMBO J., 3:2437-2442). OmpA is a major protein of the E. coli outer membrane and thus its signal peptide is thought to be efficient in the translocation process. Also, the OmpA signal peptide does not need to be modified before processing as may necessary for other signal peptides, such as, for example the lipoprotein signal peptide (G. Duffaud et al. (1987) Methods in Enzymology 153:492).

The following examples are presented by way of illustration, not by way of limitation.

### EXPERIMENTAL

### EXAMPLE 1

### Expression and Purification of AtXRCC3

For expression of AtXRCC3 in bacteria, the AtXRCC3 gene was ligated into the bacterial expression vector pBAD/gIIIA (Invitrogen Corp., Carlsbad, Calif.) and electroporated in competentent TOP10 E. coli cells (Invitrogen Corp., Carlsbad, Calif.). A single recombinant E. coli colony was used to inoculate 250 mL of LB medium containing 50 ug/mL ampicillin which was grown at 37° C. Protein production was induced at log phase by adding 0.002% arabinose. After an incubation of 6 hr, the cells were harvested by centrifugation at 8,000×g. Protein extraction was carried out according to Qiagen “batch purification under denaturing conditions” protocol. Samples were resuspended in 5 mL/g of lysis buffer: 100 mMNaH2PO4, 10 mM Tris-Cl, 6M GuHCl, pH 8.0, and incubated at RT for 1 hour following centrifugation at 10,000×g for 30 min. The cleared lysate was bound to Ni-NTA resin (Qiagen, Valencia, Calif., Cat. #30210) for 40 min. at RT with gentle agitation. The column was washed with 20 bed volumes of 1 00 mMNaH₂PO₄, 10 mM Tris-Cl, 8 M Urea, 10% glycerol, 15 mM β-mercaptoethanol adjusted to pH 6.3. Recombinant protein was collected with 0.5 ml of elution buffer (as above but pH 5.5, and pH 4.5). Purified XRCC3 was dialyzed using a linear 6M-1M urea gradient in 50 mM Tris-HCl-7.5, 50 mM NaCl, 15 mM β-mercaptoethanol, 10% glycerol containing protease inhibitors and stored at −80° C.

### EXAMPLE 2

### Production of Antibodies Directed Against AtXRCC3

Purified protein was subjected to SDS-PAGE electrophoresis and AtXRCC3 was excised from the gels and used for the production of rabbit anti-AtXRCC3 antibodies (COVANCE Research Products, Inc). Antibody titer and specificity were determined through western analysis using standard procedures. Further purification of anti-AtXRCC3 antibodies was performed using an ImmunoPure IgG (Protein A) Purification Kit (Pierce) according to the manufacturer's recommendations.

### EXAMPLE 3

### AtXRCC3 Has ATPase and GTPase Activities

The ATPase activity of AtXRCC3 was measured by using an activity assay which employed gamma ³²P-ATP (NEN LifeSciences Products)(6 mCi). Renatured XRCC3 protein was produced as described in Example 1, and 200 ng was added to gamma ³²P-ATP (0.5 μCi), ATPase buffer (20 mM Tris-HCl, pH 7.5, 8 mM MgCl₂, 0.1 mM DTT, 50 mM KCl, 2% glycerol, 50 μg/mL BSA), and, where indicated, 50 ng of ssDNA (M13mp19ssDNA) or 100 ng dsDNA (M13mp19RF) were added in a 25 μL reaction volume, and incubated at 30° C. for 0, 15, 30, 60, or 90 minutes. Fifteen μL aliquots of each reaction were electrophoresed through a 20% TBE gel at 150 V for 30 min. Gels were exposed to a Phosphoimager for 1 hr, and quantified by using ImageQuant software. GTPase assays were performed as above by substituting gamma ³²P-GTP for the gamma ³²P-ATP.

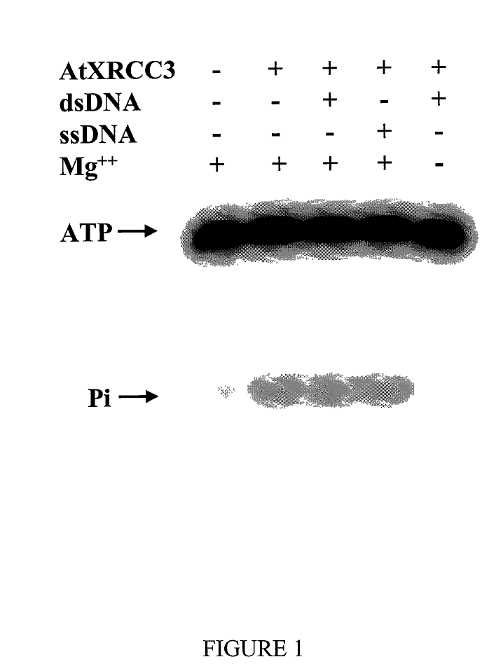

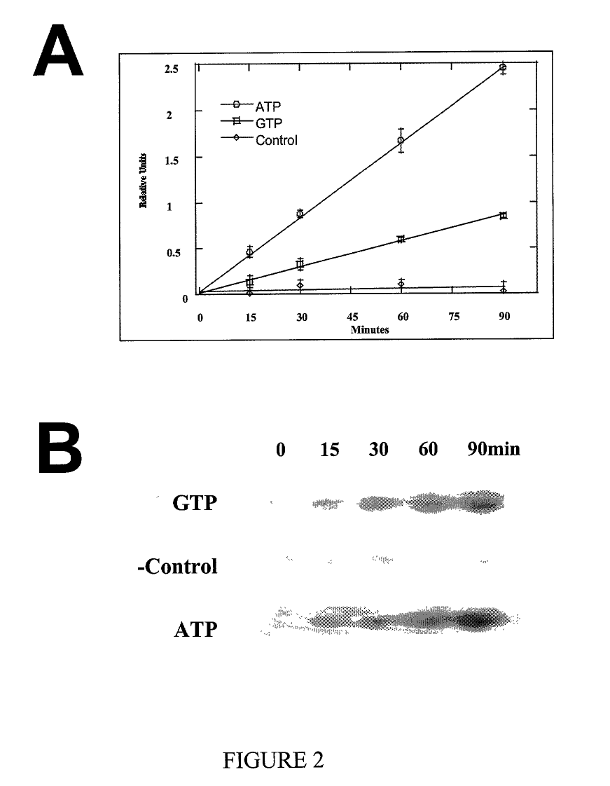

The results of the ATPase assays are depicted in FIGS. 1 and 2. The results of the GTPase assays are depicted in FIG. 1. The results of the assays reveal that AtXRCC3 has both ATPase and GTPase activities. FIG. 1 illustrates that ATPase activity of AtXRCC3 is dependent on the presence of Mg²⁺, which is typical for ATPases. The presence of single or double-stranded DNA had no effect on the AtXRCC3's ATPase activity. The upper panel in FIG. 2 illustrates the results of a time course of the ATPase and GTPase activity of AtXRCC3. The figure indicates the production of ³²P FIG. 2 also indicates that AtXRCC3 has a substrate preference for ATP over GTP. The bottom panel of FIG. 2 illustrates the free phosphate cleavage products from the ATPase and GTPase reactions.

### EXAMPLE 4

### Immunolocalization of AtXRCC3 in UV-Treated and Untreated Suspension-Cultured Arabidopsis Cells

Arabidopsis suspension-cultured cells were UV (ultra violet) irradiated (8,000 J/m2), and collected 24 hrs. after treatment. Untreated suspension cells were used as a control. Treated and untreated cells were fixed in 3.7% formaldehyde, 5% DMSO in PHEM buffer pH 6.9 for 1 hr at room temperature. The fixative was removed with three washes of PHEM buffer pH 6.9. The cells were then aliquoted on a micro-cover glass which was sealed with 0.75% agar in water. The cell walls were digested with 1% cellulase for 10 min and then washed three times with PHEM buffer pH 6.9, and once with 1% Triton X-100. Blocking was accomplished with 3% BSA in PHEM pH 6.9 for 2 hrs., followed by incubation with primary antibody: no antibody, pre-immune or anti-XRCC3 antibody overnight at 4° C. Unbound antibodies were remove with five washes of PHEM pH 6.9. The cells were incubate with the secondary antibody (goat-anti-rabbit-FITC conjugated) for,2 hrs. at room temperature. Nuclear staining was conducted with propidium iodide for 30 min. The cells were mounted with 20% MOWIOL, 0.1% phenelendiamine in PBS-9.0.

The results of the inmmunolocalization experiments revealed that AtXRCC3 protein expression is UV-inducible in Arabidopsis suspension-cultured cells, and further that AtXRCC3 is associated with condensed chromosomes in UV-induced cells but not in control cells that were not subjected to UV irradiation.

### EXAMPLE 5

### Inmunolocalization of AtXRCC3 in UV-Treated and Untreated Arabidopsis Flowers

Flowering Arabidopsis plants were UV irradiated (8,000 J/m2) and collected 24hrs. after treatment. Arabidopsis seeds were germinated on MSO media, and roots were UV-radiated under the above conditions. Tissues were fixed in 4% Paraformaldehyde, 5% DMSO, 0.01% Triton X-100 in PHEM buffer pH 6.9 by vacuum for 2 hr. The fixative solution was removed with three washes with PHEM buffer pH 6.9, following dehydration with: 10, 30, 50, 70, 90, 100% EtOH/30min. Embedding of the tissue began with: 2 parts EtOH/1 part LR White Resin×1 hr, 1 part EtOH/2 parts LR White Resin×2 hrs., and finally 100% LR White Resin with an overnight incubation at 4° C. Embedded flower tissue was placed into gelatin capsules containing LR White Resin, and root tips were excised under prior to embedding. Polymerization was carried out in a 50° C. incubator for 16 hrs. Longitudinal sections were of 1 μm in thickness.

The results of these inmmunolocalization experiments revealed that AtXRCC3 protein is present in floral tissues following UV-irradiation. AtXRRCC3 was not detected in floral tissue of control plants that had not been UV-irradiated.

### EXAMPLE 6

### Transformation and Regeneration of Transgenic Maize Plants

Immature maize embryos from greenhouse donor plants are bombarded with a plasmid containing an XRCC3 nucleotide sequence of the invention operably linked to a maize ubiquitin plus a plasmid containing the selectable marker gene PAT (Wohlleben et al. (1988) Gene 70:25-37) that confers resistance to the herbicide Bialaphos. Transformation is performed as follows. Media recipes follow below.

Preparation of Target Tissue

The ears are surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Bombardment and Culture Media

Bombardment medium (560Y) comprises 4.0 g/1 N6 basal salts (SIGMA C-1416), 1.0 ml/L Eriksson's Vitamin Mix (1000X SIGMA-1511), 0.5 mg/L thiamine HCl, 120.0 g/L sucrose, 1.0 mg/L 2,4-D, and 2.88 g/L L-proline (brought to volume with D-I H₂O following adjustment to pH 5.8 with KOH); 2.0 g/L Gelrite (added after bringing to volume with D-I H₂O); and 8.5 mg/L silver nitrate (added after sterilizing the medium and cooling to room temperature). Selection medium (560R) comprises 4.0 g/L N6 basal salts (SIGMA C-1416), 1.0 ml/L Eriksson's Vitamin Mix (1000X SIGMA-1511), 0.5 mg/L thiamine HCl, 30.0 g/L sucrose, and 2.0 mg/L 2,4-D (brought to volume with D-I H₂O following adjustment to pH 5.8 with KOH); 3.0 g/L Gelrite (added after bringing to volume with D-I H₂O); and 0.85 mg/L silver nitrate and 3.0 mg/L bialaphos (both added after sterilizing the medium and cooling to room temperature).

Plant regeneration medium (288J) comprises 4.3 g/L MS salts (GIBCO 11117-074), 5.0 ml/L MS vitamins stock solution (0.100 g nicotinic acid, 0.02 g/L thiamine HCl, 0.10 g/l pyridoxine HCL, and 0.40 g/L glycine brought to volume with distilled D-I H₂O) (Murashige and Skoog (1962) Physiol. Plant. 15:473), 100 mg/1 myo-inositol, 0.5 mg/L zeatin, 60 g/L sucrose, and 1.0 ml/L of 0.1 mM abscisic acid (brought to volume with polished D-I H₂O after adjusting to pH 5.6); 3.0 g/L Gelrite (added after bringing to volume with D-I H₂O); and 1.0 mg/L indoleacetic acid and 3.0 mg/L bialaphos (added after sterilizing the medium and cooling to 60° C.). Hormone-free medium (272V) comprises 4.3 g/L MS salts (GIBCO 11117-074), 5.0 ml/L MS vitamins stock solution (0.100 g/L nicotinic acid, 0.02 g/L thiamine HCL, 0.10 g/L pyridoxine HCl and 0.40 g/L glycine brought to volume with polished D-I H₂O), 0.1 g/L myo-inositol, and 40.0 g/L sucrose (brought to volume with polished D-I H₂O after adjusting pH to 5.6); and 6 g/L bacto-agar (added after bringing to volume with polished D-I H₂O), sterilized and cooled to 600° C.

Preparation of DNA

A plasmid vector comprising the XRCC3 operably linked to a maize ubiquitin is made. This plasmid DNA plus plasmid DNA containing a PAT selectable marker is precipitated onto 1.1 μm (average diameter) tungsten pellets using a CaCl₂ precipitation procedure as follows:

100 μL prepared tungsten particles in water

10 μL (1 μg) DNA in TrisEDTA buffer (1 μg total)

100 μL 2.5 M CaCl₂

1.0 μL 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 mL 100% ethanol, and centrifuged for 30 seconds. Again the liquid is removed, and 105 μL 100% ethanol is added to the final tungsten particle pellet. For particle gun bombardment, the tungsten/DNA particles are briefly sonicated and 10 μL spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for XRCC3 biological activity.

### EXAMPLE 7

### Agrobacterium-mediated Transformation and Regeneration of Transgenic Maize Plants

For Agrobacterium-mediated transformation of maize with an XRCC3 nucleotide sequence of the invention, preferably the method of Zhao is employed (PCT patent publication WO98/32326), the contents of which are hereby incorporated by reference. Briefly, immature embryos are isolated from maize and the embryos contacted with a suspension of Agrobacterium, where the bacteria are capable of transferring the XRCC3 nucleotide sequence of interest to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants.

### EXAMPLE 8

### Production of Transgenic Soybean Plants Using Embryo Transformation

Soybean embryos are bombarded with a plasmid containing XRCC3 nucleotide sequence of the invention operably linked to a SCP1 or UCP3 promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No.4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the XRCC3 nucleotide sequence of the invention operably linked to the SCP1 or UCP3 promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂(2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### EXAMPLE 9

### Production of Transgenic Sunflower Plants Using Meristem Tissue Transformation

Sunflower meristem tissues are transformed with an expression cassette containing an XRCC3 nucleotide sequence of the invention operably linked to a SCP1 promoter as follows (see also European Pat. Number EP 0 486233, herein incorporated by reference, and Malone-Schoneberg et al. (1994) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer et al. (Schrammeijer et al.(1990) Plant Cell Rep. 9: 55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige et al. (1962) Physiol. Plant., 15: 473-497), Shepard's vitamin additions (Shepard (1980) in Emergent Techniques for the Genetic Improvement of Crops (University of Minnesota Press, St. Paul, Minn.), 40 mg/l adenine sulfate, 30 g/l sucrose, 0.5 mg/16-benzyl-aminopurine (BAP), 0.25 mg/l indole-3-acetic acid (IAA), 0.1 mg/l gibberellic acid (GA₃), pH 5.6, and 8 g/l Phytagar.

The explants are subjected to microprojectile bombardment prior to Agrobacterium treatment (Bidney et al. (1992) Plant Mol. Biol. 18: 301-313). Thirty to forty explants are placed in a circle at the center of a 60×20 mm plate for this treatment. Approximately 4.7 mg of 1.8 μm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the XRCC3 nucleotide sequence of the invention operably linked to the SCP1 promoter is introduced into Agrobacterium strain EHA105 via freeze-thawing as described by Holsters et al. (1978) Mol Gen. Genet. 163:181-187. This plasmid further comprises a kanamycin selectable marker gene (i.e, nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for XRCC3 activity as described supra.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with Parafilm to secure the shoot. Grafted plants can be transferred to soil following one week of in vitro culture. Grafts in soil are maintained under high humidity conditions followed by a slow acclimatization to the greenhouse environment. Transformed sectors of To plants (parental generation) maturing in the greenhouse are identified by NPTII ELISA and/or by XRCC3 activity analysis of leaf extracts while transgenic seeds harvested from NPTII-positive T₀ plants are identified by XRCC3 activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox bleach solution with the addition of two to three drops of Tween 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA 105 via freeze thawing as described previously. The pellet of overnight-grown bacteria at 28° C. in a liquid YEP medium (10 g/l yeast extract, 10 g/l Bactopeptone, and 5 g/l NaCl, pH 7.0) in the presence of 50 μg/l kanamycin is resuspended in an inoculation medium (12.5 mM 2-(N-morpholino) ethanesulfonic acid, MES, 1 g/l NH₄Cl and 0.3 g/l MgSO₄ at pH 5.7) to reach a final OD6₀₀ of 4.0. Particle-bombarded explants are transferred to GBA medium (374E), and a droplet of bacteria suspension is placed directly onto the top of the meristem. The explants are co-cultivated on the medium for 4 days, after which the explants are transferred to 374C. medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C. medium are screened for expression of the selectable marker gene and then those that are positive for expression of the marker gene are then screened for XRCC3 activity using assays known in the art. After positive (i.e., for XRCC3 expression) explants are identified, and every positive explant is subdivided into nodal explants. One nodal explant contains at least one potential node. The nodal segments are cultured on GBA medium for three to four days to promote the formation of auxiliary buds from each node. Then they are transferred to 374C. medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for XRCC3 expression are grafted to Pioneer hybrid 6440 in vitro-grown sunflower seedling rootstock. The rootstocks are prepared in the following manner. Seeds are dehulled and surface-sterilized for 20 minutes in a 20% Clorox bleach solution with the addition of two to three drops of Tween 20 per 100 ml of solution, and are rinsed three times with distilled water: The sterilized seeds are germinated on the filter moistened with water for three days, then they are transferred into 48 medium (half-strength MS salt, 0.5% sucrose, 0.3% gelrite pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

That which is claimed:

1. An isolated polypeptide comprising an amino acid sequence selected from the group consisting of:
(a) the amino acid sequence set forth in SEQ ID NO: 2;
(b) the amino acid sequence encoded by SEQ ID NO: 1;
(c) an amino acid sequence comprising at least 100 contiguous amino acids of the amino acid sequence set forth in SEQ ID NO: 2; and
(d) an amino acid sequence comprising at least 80% identity to the amino acid sequence set forth in SEQ ID NO: 2;
wherein said polypeptide comprises XRCC3 activity.

2. An isolated nucleic acid molecule comprising a member selected from the group consisting of:
(a) the nucleotide sequence set forth in SEQ ID NO: 1;
(b) a nucleotide sequence encoding the amino acid sequence set forth in SEQ ID NO: 2;
(c) a nucleotide sequence comprising at least 80% sequence identity to the nucleotide sequence set forth in SEQ ID NO: 1;
(d) a nucleotide sequence comprising at least 100 contiguous nucleotides of the nucleotide sequence set forth in SEQ ID NO: 1;
(e) a nucleotide sequence that hybridizes to the nucleotide sequence set forth in SEQ ID NO: 1 under hybridization conditions comprising (i) hybridization in a solution comprising formamide at a concentration of 40 to 45%, 1.0 M NaCl, 1% SDS, and a temperature of about 37° C., and (ii) at least one post hybridization wash in a solution comprising 0.5X to 1X SSC and a temperature of 55 to 60° C.; and
(f) a nucleotide sequence that is complementary to the nucleotide sequence of (a), (b), (c), (d), or (e);
wherein said member encodes a polypeptide comprising XRCC3 activity or said member is complementary to a nucleotide sequence that encodes a polypeptide comprising XRCC3 activity.

3. An expression cassette comprising a promoter operably linked to the nucleotide sequence of claim 2.

4. The expression cassette of claim 3, wherein said promoter drives expression in a plant.

5. A non-human host cell comprising in its genome the expression cassette of claim 3.

6. The host cell of claim 5, wherein said host cell is selected from the group consisting of a plant cell, an animal cell, a yeast cell, and a bacterial cell.

7. A transformed plant comprising in its genome at least one stably incorporated nucleotide construct comprising a promoter that drives expression in a plant operably linked to a nucleotide sequence comprising a member selected from the group consisting of:
(a) the nucleotide sequence set forth in SEQ ID NO: 1;
(b) a nucleotide sequence encoding the amino acid sequence set forth in SEQ ID NO: 2;
(c) a nucleotide sequence comprising at least 80% sequence identity to the nucleotide sequence set forth in SEQ ID NO: 1;
(d) a nucleotide sequence comprising at least 100 contiguous nucleotides of the nucleotide sequence set forth in SEQ ID NO: 1;
(e) a nucleotide sequence that hybridizes to the nucleotide sequence set forth in SEQ ID NO: 1 under hybridization conditions comprising (i) hybridization in a solution comprising formamide at a concentration of 40 to 45%, 1.0 M NaCl, 1% SDS, and a temperature of about 37° C., and (ii) at least one post hybridization wash in a solution comprising 0.5X to 1X SSC and a temperature of 55 to 60° C.; and
(f) a nucleotide sequence that is complementary to the nucleotide sequence of (a), (b), (c), (d), or (e);
wherein said member encodes a polypeptide comprising XRCC3 activity or said member is complementary to a nucleotide sequence that encodes a polypeptide comprising XRCC3 activity.

8. The plant of claim 7, wherein said promoter is selected from the group consisting of constitutive, tissue-preferred, and chemical-regulated promoters.

9. The plant of claim 7, wherein said plant is a monocot.

10. The plant of claim 9, wherein said monocot is selected from the group consisting of maize, wheat, rice, sorghum, barley, millet, and rye.

11. The plant of claim 7, wherein said plant is a dicot.

12. The plant of claim 11, wherein said dicot is selected from the group consisting of tobacco, tomato, potato, soybean, Brassica sp., alfalfa, safflower, sunflower, cotton, peanut, and Arabidopsis.

13. Transformed seed of the plant of claim 7.

14. A method for altering recombination frequency in a plant comprising introducing into a plant a nucleotide sequence comprising a member selected from the group consisting of:
(a) the nucleotide sequence set forth in SEQ ID NO: 1;
(b) a nucleotide sequence encoding the amino acid sequence set forth in SEQ ID NO: 2;
(c) a nucleotide sequence comprising at least 80% sequence identity to the nucleotide sequence set forth in SEQ ID NO: 1;
(d) a nucleotide sequence comprising at least 100 contiguous nucleotides of the nucleotide sequence set forth in SEQ ID NO: 1;
(e) a nucleotide sequence that hybridizes to the nucleotide sequence set forth in SEQ ID NO: 1 under hybridization conditions comprising (i) hybridization in a solution comprising formamide at a concentration of 40 to 45%, 1.0 M NaCl, 1% SDS, and a temperature of about 37° C., and (ii) at least one post hybridization wash in a solution comprising 0.5X to 1X SSC and a temperature of 55 to 60° C.; and
(f) a nucleotide sequence that is complementary to the nucleotide sequence of (a), (b), (c), (d), or (e);
wherein said member: encodes a polypeptide comprising XRCC3 activity, encodes a dominant-negative XRCC3 protein, or is complementary to a nucleotide sequence that encodes a polypeptide comprising XRCC3 activity; and wherein said recombination frequency is increased or decreased in said plant or at least one cell thereof.

15. The method of claim 14, wherein said nucleotide construct further comprises a promoter that drives expression in a plant cell, said promoter operably linked to said nucleotide sequence.

16. The method of claim 15, wherein said promoter is operably linked to said nucleotide sequence for the production of antisense transcripts.

17. The method of claim 15, wherein said promoter is selected from the group consisting of constitutive, tissue-preferred, and chemical-regulated promoters.

18. The method of claim 15, wherein said plant or at least one cell thereof comprises a dominant-negative phenotype.

19. The method of claim 15 further comprising antisense suppression or co-suppression.

20. The method of claim 14 further comprising chimeraplasty.


SEQUENCE LISTING (2 sequences)

SEQ ID NO: 1 - DNA, 915 bp, Arabidopsis thaliana
  CDS (1)..(912)
atgcaaaatgggaaaattaagccggagaatctcctccgacgatcgccgacgaatcgtaaactaaccacaggctgtgaaatcctcgacggatgtcttcgcggcggaatttcctgcgattctttaaccgaaatcgttgcggagagtggctgcggaaagacacagctctgccttcagctttcgctctgtacccaacttccgatttcacacggcggactcaacggttcttctctttatctccattcagagttcccatttccgtttcgtcgtctccatcagctctcgcacacgtttcatcaatcaaaccctagtatttacgcgaattacaatgataatccttgtgatcatgtgtttgtacaaaatgtccattctgtagaccacctgttcgacataatgcctaggatagatgggtttgttgggaattcgaaaactaggtttccgttgaagttgattgtgcttgattctgtagcggcattgtttagatcggagtttgataatactccgtctgatcttaagaagagatcttctttgtttttcaagatatctgggaaattgaagcagttggctagcaagtttgatttggctattgtgattacgaatcaggttacggatttggttgaaacttctgatgggttaagtgggctgaggattgggaatttgaggtatttgtattcgtctggaagacgagttgttccttctttaggcttggcttgggctaattgtgtgaattccagattttttatttcacgaagtgatggtagtattgttaaagatagaagtgaaaaggacgagaattgttcaagttctgtgtccagaagtgctaagagacggcttgacatagtgttttcaccttatttgcctgggagttcttgcgagttcatgatcacacgagaggggatttgtgcggttcaagcgtag

SEQ ID NO: 2 - protein, 304 aa, Arabidopsis thaliana
MQNGKIKPENLLRRSPTNRKLTTGCEILDGCLRGGISCDSLTEIVAESGCGKTQLCLQLSLCTQLPISHGGLNGSSLYLHSEFPFPFRRLHQLSHTFHQSNPSIYANYNDNPCDHVFVQNVHSVDHLFDIMPRIDGFVGNSKTRFPLKLIVLDSVAALFRSEFDNTPSDLKKRSSLFFKISGKLKQLASKFDLAIVITNQVTDLVETSDGLSGLRIGNLRYLYSSGRRVVPSLGLAWANCVNSRFFISRSDGSIVKDRSEKDENCSSSVSRSAKRRLDIVFSPYLPGSSCEFMITREGICAVQA